(19) United States
(12) Patent Application Publication
(10) Pub. No.: US 2001/0041882 A1
(43) Pub. Date: Nov. 15, 2001

(54) FEMALE URINARY DEVICE

(76) Inventor: GILBERT B. BRINK, ALHAMBRA, CA (US)

Correspondence Address: BONIARD I BROWN, 1500 WEST COVINA PARKWAY 113, WEST COVINA, CA, 917902793

(21) Appl. No.: 09/458,733

(22) Filed: Dec. 13, 1999

(51) Int. Cl. A61M 27/00

(52) U.S. Cl. 604/544

(57) ABSTRACT

A catheter for urinary relief of relatively incapacitated female persons has a head portion with a rim and flange to sealingly engage tissue about a urethra and an outlet tube. A slotted urinal cap facilitates entry of the tube into a urinal.

### BACKGROUND AND SUMMARY OF THE INVENTION

A need has existed for an effective urinary relief device for urination by disabled, bedridden, or otherwise incapacitated or handicapped female persons.

Devices of the prior art do not address or adequately meet the needs of such persons. Some prior art devices are relatively complicated and/or expensive, and some are difficult or inconvenient to utilize by such persons.

The device of Ellis U.S. Pat. No. 3,512,185 is relatively complicated and somewhat intricate and expensive to fabricate. It requires an additional component or tube to conduct any urine to a urinal for disposal.

Wapner U.S. Pat. No. 4,911,689 is relatively complicated, expensive, and involves a block member for insertion into a vaginal opening, which is unnecessary for purposes of the present invention. It is adapted for collection of specimens free of vaginal or anal bacteria, and lacks any tube or the like for conducting urine to disposal or to a urinal. This outlet is so directed as to not be useful for the purposes of the present invention.

Davis U.S. Pat. No. 3,555,102 provides structure for insertion into a vaginal cavity, but lacks means for closely engaging tissues about a urethral opening for prevention of leakage.

The present invention provides a urinary relief device for female persons who are handicapped and/or bedridden, to facilitate urination and disposal.

The invention is an intra-labial catheter, with a head portion with a rim of generally oval configuration to engage the urethra and soft tissue thereabout for prevention of leakage, the head portion including a flange portion extending outwardly from the inward portion of the oval rim for engagement with the tissues of a person for leakage prevention. A generally funnel-shaped reservoir portion extends from the head portion to regulate dynamic flow, and a tube portion is connected with the reservoir portion for disposal of the urine. A detachable cover for an associated urinal is slitted to facilitate insertion of the tube therethrough.

### BRIEF DESCRIPTION OF THE DRAWINGS

FIG. 1 is an elevational view, partially in section, showing (1) a device of the invention in relation to the tissues of a person, shown in section, and (2) a urinal having a cap according to the invention;

FIG. 2 is a perspective view of the device of the invention shown in FIG. 1;

FIG. 3 is a partial elevational view showing details of a head portion of the device of the invention;

FIG. 4 is a sectional view of the device of FIGS. 1 and 2; and

FIG. 5 is an elevational view of the cap shown in Figure

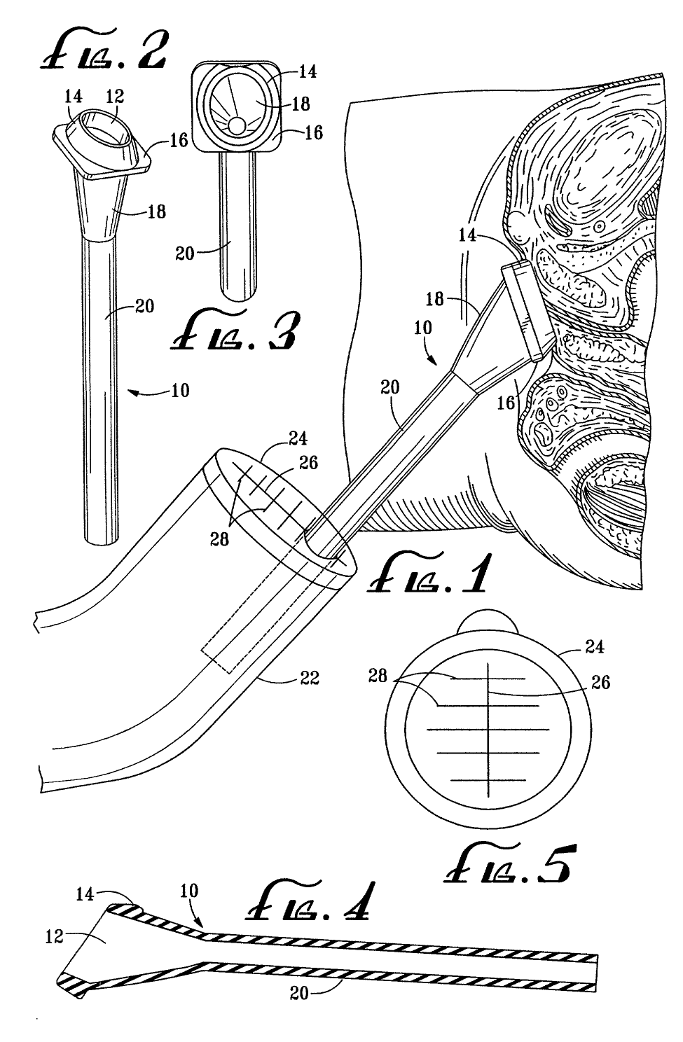

### DESCRIPTION OF THE PREFERRED EMBODIMENTS

The present invention relates to improved intra-labial catheters for urinary relief of bed-ridden or otherwise handicapped females, and further to a urinal adapter for cooperation with a catheter device for easy use by impaired females.

Referring to the drawings, a urinary relief device of the invention comprises an intra-labial catheter which has a generally oval intake opening 12 defined by a cup portion 14 adapted to be disposed between the labia and to cover the urethra of the person, and to engage the soft tissues of the person and prevent leakage with gentle pressure of the device relative to the person's soft tissues. A head portion 14 has a rim having the general configuration of an eye cup, and defining a generally oval opening about 11/16″ wide and 15/16″ long and about 3/32″ thick at its rounded edge. It is oriented or inclined relative to an adjacent funnel portion 18, as shown. Cup portion 14 is tapered in configuration to be about 3/16″ thick at its outer edge and 5/16″ at its inner portion. A transversely or laterally extending flange 16 is disposed about the base of head portion 14, and upon gentle application to the tissues of the person, facilitates improved sealing engagement of the device relative to the soft tissues including the labia minora and the urethral orifice. The device thus engages with the soft tissues of the person, with only gentle pressure, and is substantial assurance against leakage. The flange 16 is rectilinear in configuration and may typically be 20/16″ wide by 23/16″.

A gemerally funnel-shaped or frusto-conical reservoir portion 18 extends from the flange 16 to regulate and serve as a temporary reservoir to maintain flow into a tube portion 20 from whence the urine flows into a urinal 22, typically disposed between the legs of the user.

A lid or cap 24 adapted for positioning at an end opening of the urinal, and for quick removal therefrom, preferably by a snap-fit arrangement, and has an elongated slit 26 intersected at right angles by a plurality of slits 28. The slits enable a person, although handicapped, to easily urge the outer end of the tube 20 of the device 10 through the cap, thereby essentially eliminating leakage between the tube and the urinal.

The tube portion 20 is sufficiently flexible to enable a limited degree of bending and changes of orientation, thus to ensure passage of urine into the urinal 22. The tube portion 20 is sufficiently transparent to enable a person, such as an attendant, nurse, or the user herself, to monitor urine flow with the female user prone, or to enable the user to monitor herself, particularly in a standing position. Utilization of the device with the person in a standing position, such as in a doctor's office or a laboratory, or at home, for the purpose of collecting a urine specimen for testing, and is also useful for female persons in unsanitary restrooms or in situations where no restroom is available, such as in remote areas.

The device 10 is fabricated of relatively soft, resilient and somewhat flexible plastic, and is preferably tinted, but translucent. The flexibility the different portions of the device vary in accordance with the thickness of the respective portions, thus reservoir 18 is relatively firm, and tube 20 has sufficiently thick walls as to prevent kinking, but thin enough to enable considerable bending for adjustment in directions, etc. Cup portion 14 is relatively soft to assist in a sealing action between it and the tissues of the person to prevent leakage.

The device adapts itself to variations in body features while held in place gently against the body of the person.

The device is preferably and typically fabricated by injection molding, and is sufficiently low in cost to be disposable after one or two uses. It is capable of withstanding sanitizing with boiling water, and ordinarily may be cleansed by washing in soapy, warm water. The orifice may be sprayed with Lysol or other disinfectant, as when the device is to be used by another or second person.

In utilization of the device with the user in bed, it may be desirable to have an opened absorbent undergarment in place, or to place a folded towel beneath the hips of the user, in case of unexpected leakage. In utilizing the device during a menstrual period, the use of a tampon is appropriate.

It will be understood that various changes and modifications may be made from the preferred embodiments discussed above without departing from the scope of the present invention, which is established by the following claims and equivalents thereof:

The inventor claims:

1. An intra-labial catheter for urinary relief of female persons, comprising:
a head portion having a rim and a flange,
said rim being of generally oval configuration and enlarged in cross-section inwardly of the device,
said flange extending radially outwardly from the inner portion edge of the rim,
said head portion rim and flange engaging about the urethra and soft tissue of the person in sealing engagement, and
a tube portion connected with the head portion for conducting urine to disposal.

2. A catheter according to claim 1, wherein said oval configuration is about 11/16″ wide and 15/16″ long.

3. A catheter according to claim 1, wherein said rim is tapered to about 3/16″ thick at its outer edge and 5/16″ at its inward base portion.

4. A catheter according to claim 1, wherein said flange is of rectilinear configuration having a width of about 20/16″ by 23/16″ long.

5. A catheter according to claim 1, wherein the catheter is formed of a relatively soft, resilient, and somewhat flexible plastic material.

6. A catheter according to claim 1, wherein:
respective portions of the catheter vary in thickness, the tube portion relatively thinner and sufficiently thick to prevent kinking thereof and sufficiently thick to enable bending for adjustment in tube orientation, and the head portion is relatively soft to facilitate sealing engagement between the device and the tissues of a person.

7. A catheter according to claim 1, wherein said rim has a dull edge to engage said soft tissue about the urethra.

8. A catheter according to claim 1, and further comprising:
a urinal for receiving urine from the catheter for disposal, and
a detachable cap having at least one slit therein for insertion therethrough of said tube portion of the catheter.

9. A device according to claim 8, wherein said cap has at least one slit, whereby said tube portion of the catheter is readily insertable into the urinal via the cap for transfer of urine without leakage.

10. A urinary relief system for female persons, comprising:
a head portion having a rim and a flange,
said rim being of generally oval configuration and enlarged in cross-section inwardly of the device,
said flange extending radially outwardly from the inner portion edge of the rim,
said head portion rim and flange engaging about the urethra and soft tissue of the person in sealing engagement,
a generally funnel-shaped reservoir portion extending from the flange portion and tapering to a reduced dimension remote from the flange portion to regulate and smooth dynamic flow, and
a tube portion extending from said reservoir portion to conduct urine to disposal.

11. A catheter according to claim 10, wherein the catheter is formed of a relatively soft, resilient, and somewhat flexible plastic material.

12. A catheter according to claim 10, wherein:
respective portions of the catheter vary in thickness, the tube portion relatively thinner and sufficiently thick to prevent kinking thereof and sufficiently thick to enable bending for adjustment in tube orientation, and the head portion is relatively soft to facilitate sealing engagement between the device and the tissues of a person.

13. A catheter according to claim 10, wherein said rim is tapered to about 3/16″ thick at its outer edge and 5/16″ at its inward base portion.

14. A catheter according to claim 10, wherein said flange is of rectilinear configuration having a width of about 20/16″ by 23/16″ long.

15. A catheter according to claim 10, wherein said rim has a dull edge to engage said soft tissue about the urethra.

16. A catheter according to claim 10, and further comprising:
a urinal for receiving urine from the catheter for disposal, and
a detachable cap having at least one slit therein for insertion therethrough of said tube portion of the catheter.

17. A device according to claim 16, wherein said cap has at least one slit, whereby said tube portion of the catheter is readily insertable into the urinal via the cap for transfer of urine without leakage.
